(19) United States
(12) Patent Application Publication
(10) Pub. No.: US 2025/0339039 A1
(43) Pub. Date: Nov. 6, 2025

(54) BLOOD PRESSURE MEASUREMENT DEVICE

(71) Applicant: OMRON HEALTHCARE Co., Ltd., Kyoto (JP)

(72) Inventors: Masaki HARADA, Kyoto (JP); Brian BRIGHAM, Kyoto (JP); Yoshihiko SANO, Kyoto (JP); Yoshihide TOKKO, Kyoto (JP); Gen SUZUKI, Kanagawa (JP)

(21) Appl. No.: 19/271,213

(22) Filed: Jul. 16, 2025

Related U.S. Application Data
(63) Continuation of application No. PCT/JP2023/038728, filed on Oct. 26, 2023.

(30) Foreign Application Priority Data
Feb. 1, 2023 (JP) 2023-014294

(51) Int. Cl. A61B 5/021; A61B 5/00; A61B 5/022

(52) U.S. Cl. CPC A61B 5/02141; A61B 5/02233; A61B 5/6831; A61B 2560/0462; A61B 2562/0247

(57) ABSTRACT

A blood pressure measurement device includes: a device body; a first cuff provided at the device body and configured to press a living body; a band provided on an outer surface side of the first cuff and wound around the living body; and a restriction member configured to restrict a relative movement between the first cuff and the band in a width direction and to allow a relative movement between the first cuff and the band in a longitudinal direction.

### CROSS-REFERENCE TO RELATED APPLICATIONS

This application is the U.S. national stage application filed pursuant to 35 U.S.C. 365(c) and 120 as a continuation of International Patent Application No. PCT/JP2023/038728, filed Oct. 26, 2023, which application claims priority to Japanese Patent Application No. 2023-014294, filed Feb. 1, 2023, which applications are incorporated herein by reference in their entireties.

### TECHNICAL FIELD

The present invention relates to a blood pressure measurement device.

### BACKGROUND ART

In recent years, blood pressure measurement devices used for measuring blood pressure are utilized as means for checking a health state not only at a medical facility but also at home. A blood pressure measurement device detects vibration of the artery wall to measure blood pressure by, for example, inflating and contracting a cuff wound around the wrist or the like of a living body and detecting pressure in the cuff by using a pressure sensor.

As disclosed in WO 2012/018029, a technique is known as a blood pressure measurement device in which a hook-and-loop fastener is provided on a surface of a cuff so that when both end portions of a cuff portion are overlapped with each other, the end portions are not displaced. As disclosed in WO 2022/111377, a technique is also known as a blood pressure measurement device in which a band is slid to adjust the band position, and then a pin disposed on a cuff is inserted into an elongate hole of the band to arrange the cuff on the band (for example, see Patent Document 2).

### CITATION LIST

### Patent Literature

Patent Document 1: WO 2012/018029; Patent Document 2: WO 2022/111377

### SUMMARY OF INVENTION

### Technical Problem

In the blood pressure measurement device described in WO 2012/018029, it is difficult to adjust wrapping strength of the cuff after the blood pressure measurement device is attached to the living body. In the blood pressure measurement device described in WO 2022/111377, the band is fixed to the back of the cuff; therefore, the problem with the blood pressure measurement device is that the cuff is fixed to the band and thus a relative positional relationship between the cuff and the band in the longitudinal direction cannot be adjusted.

Thus, an object of the present invention is to provide a blood pressure measurement device that can easily adjust the relative position between a cuff and a band and can prevent the cuff from being displaced in the width direction.

### Solution to Problem

According to an aspect, a blood pressure measurement device is provided that includes: a device body; a first cuff provided at the device body and configured to press a living body; a band provided on an outer surface side of the first cuff and wound around the living body; and a restriction member configured to restrict a relative movement between the first cuff and the band in a width direction and to allow a relative movement between the first cuff and the band in a longitudinal direction.

According to this aspect, the restriction member restricts the relative movement between the band and the first cuff in the width direction and allows the relative movement between the first cuff and the band in the longitudinal direction. In the blood pressure measurement device, when the blood pressure measurement device is attached to the living body and when the first cuff is inflated, the first cuff and the band are not displaced in the width direction, and the relative position between the first cuff and the band in the longitudinal direction can be easily adjusted. Therefore, the blood pressure measurement device can suitably compress the living body by inflation of the first cuff, and thus high measurement accuracy is achieved.

In the blood pressure measurement device according to the aspect described above, the restriction member includes: a sheet fixed to the first cuff on a side close to the device body, the sheet being separable from a distal end side of the first cuff; fixing members provided at a portion in which the sheet and the first cuff face each other, the fixing members being configured to fix the sheet and the first cuff; and a first restriction portion provided on the band and into which the sheet is insertable, the first restriction portion being configured to restrict movement of the sheet in a width direction.

According to this aspect, the blood pressure measurement device can restrict the movement of the first cuff in a direction away from the band by fixing the sheet inserted into the first restriction portion and the first cuff with the fixing members.

In the blood pressure measurement device according to the aspect, the sheet, the fixing members, and the first restriction portion are formed to have lengths and/or positions that allow the sheet to be inserted into the first restriction portion and allow the sheet and the first cuff to be fixed by the fixing members when the band is wrapped around the living body having a minimum circumferential length and a maximum circumferential length and to which the device is assumed to be attached.

According to this aspect, even when the blood pressure measurement device is attached to the living body to which the device is assumed to be attached, the sheet inserted into the first restriction portion can be fixed to the first cuff. Therefore, high measurement accuracy of blood pressure can be achieved on the living body to which the device is assumed to be attached.

The blood pressure measurement device according to the aspect described above includes a second cuff that can be overlapped with the first cuff on an outer side of the first cuff. The restriction member restricts a relative movement between the second cuff and at least one of the first cuff or the band in the width direction.

According to this aspect, when the blood pressure measurement device is attached to the living body and when the first cuff and the second cuff are inflated, the first cuff, the second cuff, and the band are not displaced in the width direction, and the relative positions among the first cuff, the second cuff, and the band in the longitudinal direction can be easily adjusted. Therefore, the blood pressure measurement device can suitably compress the living body by inflation of the first cuff and the second cuff, and thus high measurement accuracy is achieved. In addition, since the first cuff and the second cuff overlap each other, the living body can be more suitably compressed by the first cuff and the second cuff overlapping each other when being inflated.

In the blood pressure measurement device according to the aspect, the restriction member includes: a sheet fixed to the first cuff on a side close to the device body, the sheet being separable from a distal end side of the first cuff; fixing members provided at a portion in which the sheet and the first cuff face each other, the fixing members being configured to fix the sheet and the first cuff; a first restriction portion provided on an inner surface of the band and into which the sheet is insertable, the first restriction portion being configured to restrict movement of the sheet in the width direction; and a second restriction member provided on an inner surface of the second cuff and into which the sheet is insertable, the second restriction portion being configured to restrict movement of the sheet in the width direction.

According to this aspect, even when the blood pressure measurement device is attached to the living body to which the device is assumed to be attached, the sheet inserted into the first restriction portion and the second restriction portion can be fixed to the first cuff. Therefore, high measurement accuracy of blood pressure can be achieved on the living body to which the device is assumed to be attached.

In the blood pressure measurement device according to the aspect, the sheet, the fixing members, the first restriction portion, and the second restriction portion are formed to have lengths and/or positions that allow the sheet to be inserted into the first restriction portion and the second restriction portion and allow the sheet and the first cuff to be fixed by the fixing members when the band is wrapped around the living body having a minimum circumferential length and a maximum circumferential length and to which the device is assumed to be attached.

According to this aspect, even when the blood pressure measurement device is attached to the living body to which the device is assumed to be attached, the sheet inserted into the first restriction portion and the second restriction portion can be fixed to the first cuff. Therefore, high measurement accuracy of blood pressure can be achieved on the living body to which the device is assumed to be attached.

### Advantageous Effects of Invention

According to the present invention, it is possible to provide a blood pressure measurement device that can easily adjust the relative position between the cuff and the band and can prevent the cuff from being displaced in the width direction.

### BRIEF DESCRIPTION OF DRAWINGS

FIG. 1 is a perspective view illustrating a configuration of a blood pressure measurement device according to an embodiment of the present invention;

FIG. 2 is a perspective view illustrating the configuration of the blood pressure measurement device;

FIG. 3 is a side view illustrating the configuration of the blood pressure measurement device;

FIG. 4 is a side view illustrating the configuration of the blood pressure measurement device in a state of being attached to a wrist;

FIG. 5 is a block diagram illustrating the configuration of the blood pressure measurement device;

FIG. 6 is a cross-sectional view illustrating a configuration of a main portion of the blood pressure measurement device;

FIG. 7 is a plan view illustrating the configuration of the main portion of the blood pressure measurement device from a rear surface side of a device body;

FIG. 8 is a plan view illustrating the configuration of the main portion of the blood pressure measurement device from the rear surface side of the device body with a part of the configuration omitted;

FIG. 9 is an exploded perspective view illustrating the configuration of the device body;

FIG. 10 is an exploded perspective view illustrating the configuration of the device body;

FIG. 11 is a block diagram illustrating an example of a fluid circuit of the blood pressure measurement device;

FIG. 12 is a perspective view schematically illustrating a configuration of a restriction member of the blood pressure measurement device;

FIG. 13 is a perspective view schematically illustrating the configuration of the restriction member of the blood pressure measurement device;

FIG. 14 is a plan view illustrating the configuration of the main portion of the blood pressure measurement device according to another embodiment of the present inventor from the rear surface side of the device body;

FIG. 15 is a side view illustrating the configuration of the blood pressure measurement device according to another embodiment of the present invention;

FIG. 16 is a plan view illustrating the configuration of the main portion of the blood pressure measurement device according to another embodiment of the present invention from the rear surface side of the device body;

FIG. 17 is a block diagram illustrating an example of the fluid circuit of the blood pressure measurement device;

FIG. 18 is a block diagram illustrating an example of the fluid circuit of the blood pressure measurement device; and,

FIG. 19 is a cross-sectional view schematically illustrating the configuration of the restriction member of the blood pressure measurement device according to another embodiment of the present invention.

### DESCRIPTION OF EMBODIMENTS

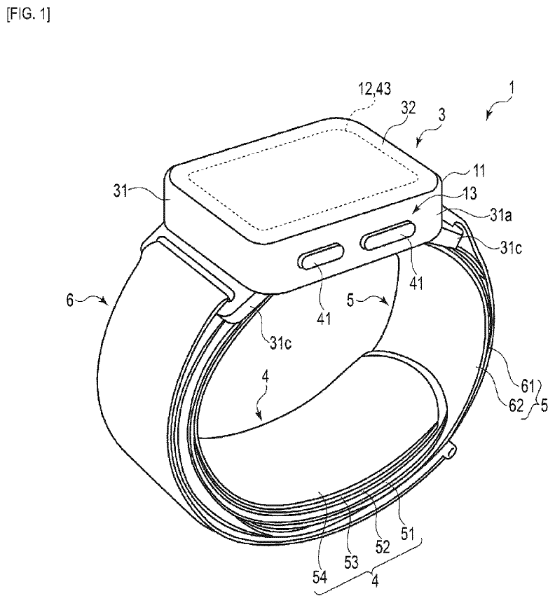

An example of a blood pressure measurement device 1 according to an embodiment of the present invention will be described below with reference to FIG. 1 to FIG. 13.

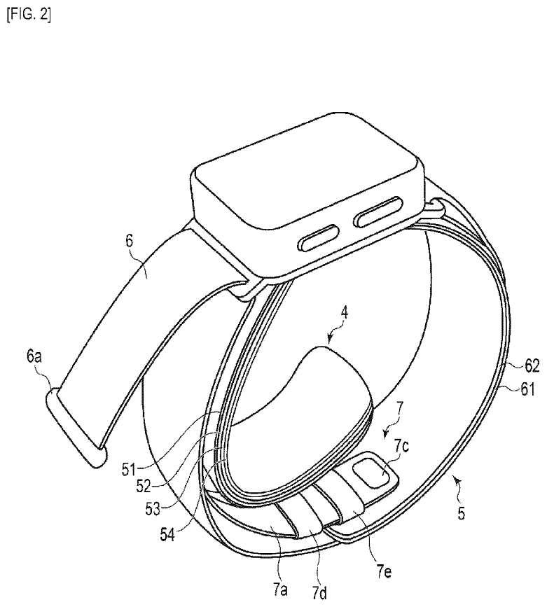

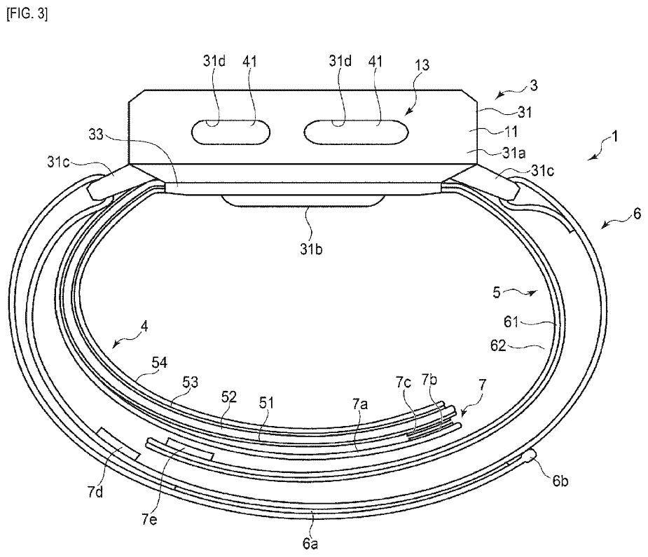

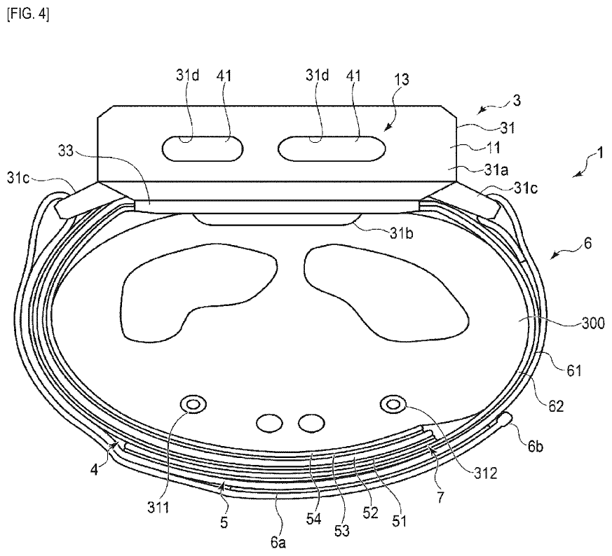

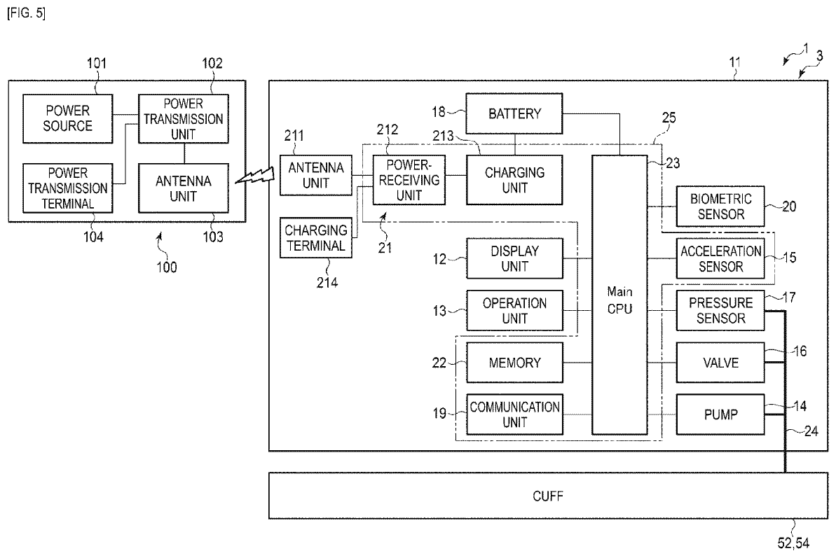

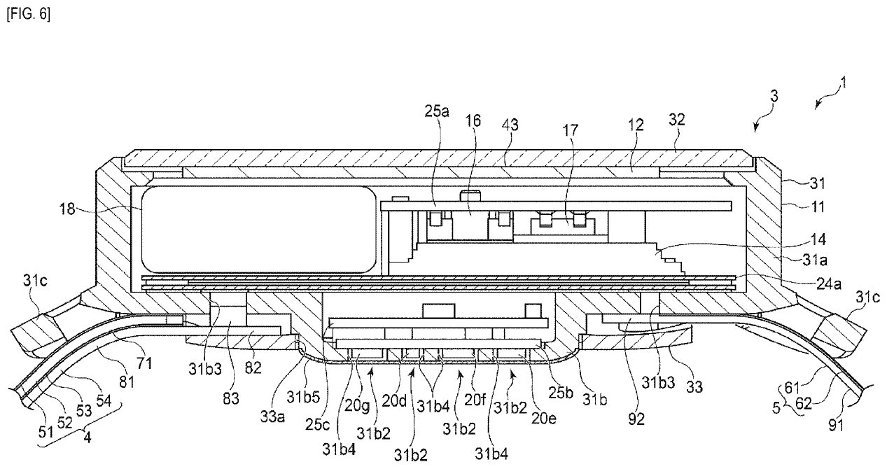

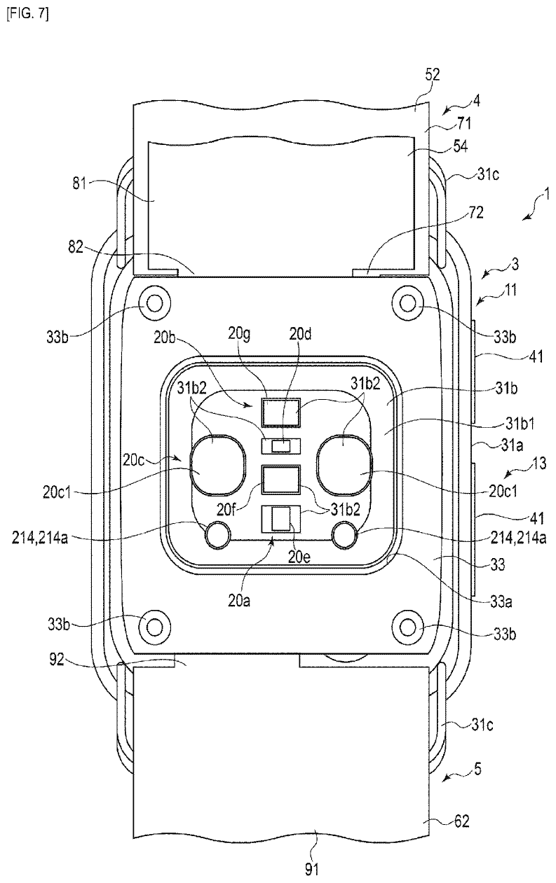

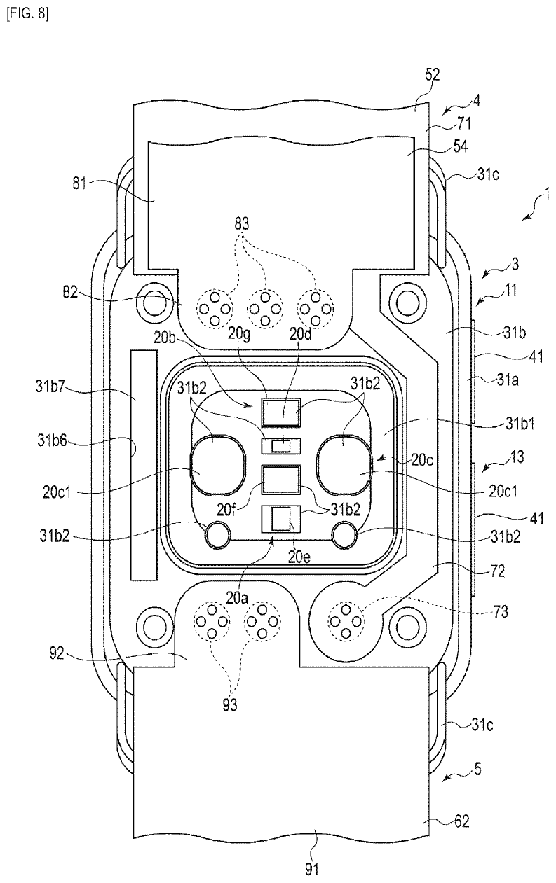

FIG. 1 is a perspective view illustrating a configuration of the blood pressure measurement device 1 according to the embodiment of the present invention. FIG. 2 is a perspective view illustrating the configuration of the blood pressure measurement device 1 in a state where a restriction member 7 is exposed. FIG. 3 is a side view illustrating the configuration of the blood pressure measurement device 1, and FIG. 4 is a side view illustrating the configuration of the blood pressure measurement device 1 in a state of being attached to a wrist 300. FIG. 5 is a block diagram illustrating the configuration of the blood pressure measurement device 1. FIG. 6 is a cross-sectional view illustrating a configuration of a device body 3, a portion of a first cuff structure 4, and a portion of a second cuff structure 5 of the blood pressure measurement device 1. FIG. 7 is a plan view illustrating the configuration of the device body 3, a portion of the first cuff structure 4, and a portion of the second cuff structure 5 of the blood pressure measurement device 1, and FIG. 8 is a plan view illustrating the configuration of the device body 3, a portion of the first cuff structure 4, and a portion of the second cuff structure 5 of the blood pressure measurement device 1 with a cuff cover 33 omitted.

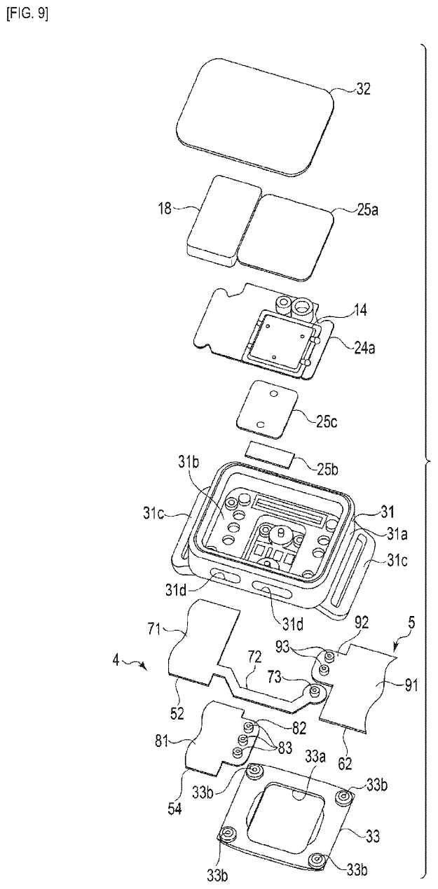

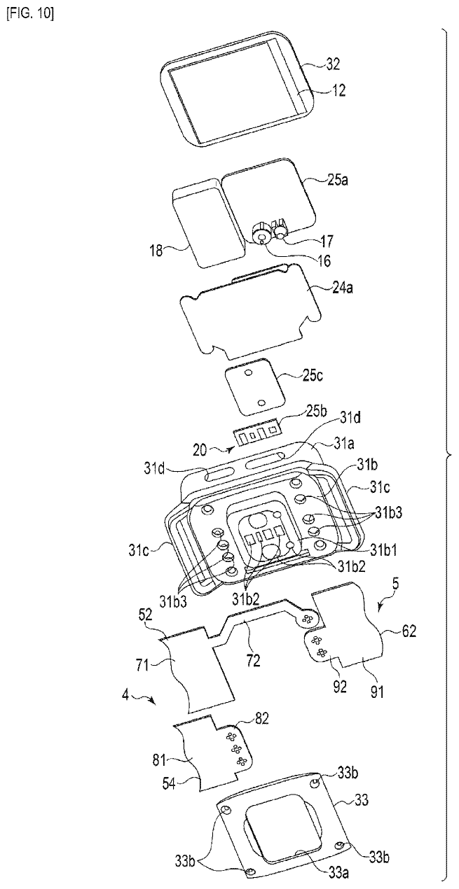

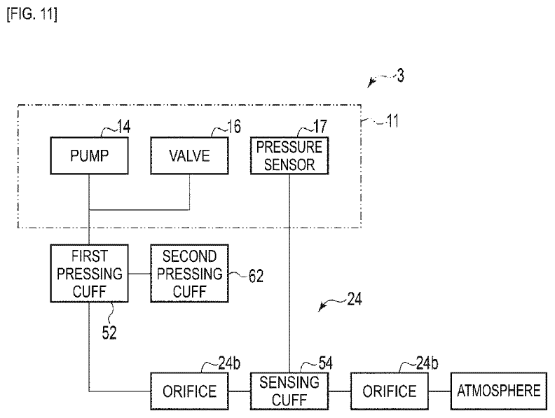

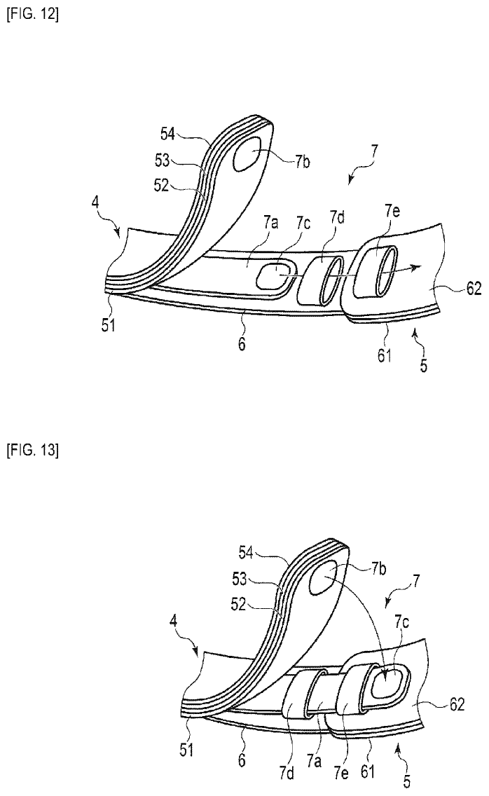

FIG. 9 is an exploded perspective view illustrating the configuration of the device body 3 from an upper surface side, and FIG. 10 is an exploded perspective view illustrating the configuration of the device body 3 from a lower surface side. FIG. 11 is a block diagram illustrating an example of a fluid circuit 24 of the blood pressure measurement device 1. FIG. 12 and FIG. 13 are perspective views each schematically illustrating a configuration of the restriction member 7 of the blood pressure measurement device 1.

As illustrated in FIG. 1 to FIG. 4, the blood pressure measurement device 1 includes, for example, the device body 3, the first cuff structure 4 (a first cuff), the second cuff structure 5 (a second cuff), a band 6, and the restriction member 7. The blood pressure measurement device 1 is configured such that the first cuff structure 4 and the second cuff structure 5 extend from the device body 3 in opposite directions of the device body 3 and the band 6 covers the first cuff structure 4 and the second cuff structure 5, and is thus formed fixable by the band 6 to the wrist 300 that is a living body. The blood pressure measurement device 1 is configured such that the restriction member 7 restricts a relative movement between the first cuff structure 4 and/or the second cuff structure 5 and the band 6 in a width direction.

As illustrated in FIG. 1 to FIG. 10, the device body 3 includes, for example, a case 11, a display unit 12, an operation unit 13, a pump 14, an acceleration sensor 15, a valve 16, a pressure sensor 17, a battery 18, a communication unit 19, biometric sensors 20, a charging circuit unit 21, a memory 22, a processor 23, the fluid circuit 24, and a substrate 25.

The case 11 is a case that accommodates components of the device body 3. The case 11 accommodates, for example, the display unit 12, the operation unit 13, the pump 14, the acceleration sensor 15, the valve 16, the pressure sensor 17, the battery 18, the communication unit 19, the biometric sensors 20, the charging circuit unit 21, the memory 22, the processor 23, the fluid circuit 24, and the substrate 25.

The case 11 includes, for example, an outer case 31, a windshield 32 that covers an upper opening of the outer case 31, and the cuff cover 33 provided below the outer case 31.

The outer case 31 is formed in a bottomed tubular shape, for example, a cylindrical shape, a rectangular tubular shape, a polygonal tubular shape, or the like having a bottom portion. In the present embodiment, an example in which the outer case 31 is formed in a bottomed rectangular tubular shape is illustrated. As a specific example, the outer case 31 includes a peripheral wall portion 31a having a rectangular tubular shape, a bottom portion 31b provided at the peripheral wall portion 31a, and a pair of loop portions 31c provided at a pair of surfaces of four outer peripheral surfaces of the peripheral wall portion 31a.

In the peripheral wall portion 31a, for example, an opening 31d in which a part of the operation unit 13 is disposed is formed. For example, the opening 31d is formed in one surface of the peripheral wall portion 31a, which is different from two surfaces provided with the pair of loop portions 31c. In addition, for example, two openings 31d are formed in one surface of the peripheral wall portion 31a.

The bottom portion 31b constitutes a rear surface (the bottom portion 31b) of the case 11 (the outer case 31). For example, a portion of the bottom portion 31b projects to be contactable with the wrist 300. For example, the bottom portion 31b includes a projection portion 31b1 obtained by forming such that a center side of an outer surface side is projected in a rectangular shape and a center side of an inner surface side of the bottom portion 31b is recessed in a rectangular shape. In addition, a plurality of window portions 31b2 in which the biometric sensors 20 are disposed and a plurality of hole portions 31b3 in which connection portions 73, 83 described below are disposed as members that fluidly connect the first cuff structure 4 and the second cuff structure 5 to the pump 14 are formed in the bottom portion 31b.

The biometric sensors 20 are disposed on the inner surface side of the projection portion 31b1. The projection portion 31b1 constitutes a sensor unit including at least one of the biometric sensors 20 or a charging terminal 214 that are disposed on the rear surface of the case 11. Here, the fact that the biometric sensors 20 and the charging terminal 214 that are disposed on the rear surface of the case 11 means that some of the components constituting the biometric sensors 20 are exposed on the rear surface of the case 11 or are disposed on the rear surface of the case 11 via a member (for example, a cover 31b5 described below) other than the first cuff structure 4 and the second cuff structure 5.

The window portions 31b2 are formed in the projection portion 31b1. For example, as illustrated in FIG. 6 to FIG. 8, the window portions 31b2 are formed by a plurality of openings 31b4 formed at a plurality of locations of the projection portion 31b1 and by the cover 31b5 having transparency, made of glass, a resin material, or the like, and covering these openings 31b4. The cover 31b5 covers, for example, an outer surface of the projection portion 31b1.

As illustrated in FIG. 10, the plurality of hole portions 31b3 are respectively formed between the projection portion 31b1 and each loop portion 31c in the bottom portion 31b. In the present embodiment, three hole portions 31b3 are formed between the projection portion 31b1 and one loop portion 31c, and other three hole portions 31b3 are formed between the projection portion 31b1 and the other loop portion 31c. These three hole portions 31b3 are aligned in one direction, for example, a direction orthogonal to a facing direction of the pair of loop portions 31c. In other words, in the bottom portion 31b that is the rear surface of the case 11, the three hole portions 31b3 to which a portion of the configuration of the first cuff structure 4 is fixed at one end portion in the one direction are disposed at positions away from the projection portion 31b1, and the three hole portions 31b3 to which another portion of the configuration of the first cuff structure 4 as well as the second cuff structure 5 are fixed at the other end portion in the one direction are disposed at positions away from the projection portion 31b1 toward the one direction.

In addition, a vent 31b6 having a slit shape long in the one direction is formed in the bottom portion 31b, and the bottom portion 31b includes a waterproof moisture-permeable sheet 31b7 covering this vent 31b6. The waterproof moisture-permeable sheet 31b7 is formed so as to prevent moisture from entering the case 11 from the outside through the vent 31b6 of the bottom portion 31b and to allow ventilation between the inside and the outside of the case 11. Here, the vent 31b6 and the waterproof moisture-permeable sheet 31b7 are provided at positions not overlapping the first cuff structure 4 and the second cuff structure 5 that are fixed to the bottom portion 31b, that is, at positions separated from the first cuff structure 4 and the second cuff structure 5. For example, in the direction orthogonal to the facing direction of the pair of loop portions 31c, a flow path body 72 of a first pressing cuff 52 described below of the first cuff structure 4 is provided adjacent to one side of the projection portion 31b1, and the vent 31b6 and the waterproof moisture-permeable sheet 31b7 are provided adjacent to the other side of the projection portion 31b1.

The loop portion 31c is formed so that the band 6 can be passed therethrough and the band 6 can be fixed thereat or the band 6 can be folded back thereat. For example, the loop portion 31c is a member having a rectangular ring shape including an opening long in one direction and through which the band 6 can be inserted. The loop portion 31c is integrally formed on an outer side surface of the outer case 31. One end of the band 6 is fixed to one loop portion 31c of the pair of loop portions 31c, and the band 6 is folded back in the other loop portion 31c.

The windshield 32 is a glass plate having a shape similar to that of an outer peripheral edge of the outer case 31, i.e., having a rectangular shape in the present embodiment. Note that the windshield 32 is not limited to a glass plate as long as the material has transparency or translucency.

The cuff cover 33 covers the bottom portion 31b of the outer case 31. In the cuff cover 33, the projection portion 31b1 of the bottom portion 31b is disposed and an opening 33a through which the projection portion 31b1 is exposed to the outside is formed. For example, the projection portion 31b1 is formed to have a thickness and into a shape projecting from a main surface of the cuff cover 33, which faces the wrist 300 when the cuff cover 33 is fixed to the bottom portion 31b. In addition, for example, the cuff cover 33 is provided with holes 33b formed at four corners and in which screws are disposed, and is detachably fixed to the bottom portion 31b of the outer case 31 of the case 11 by the screws or the like.

The cuff cover 33 covers end portions of the first cuff structure 4 and the second cuff structure 5, which are disposed on the bottom portion 31b, and thus the end portions of the first cuff structure 4 and the second cuff structure 5 are disposed in gaps generated between the bottom portion 31b and the cuff cover and the first cuff structure 4 and the second cuff structure 5 are fixed to the bottom portion 31b. As a specific example, the cuff cover 33 covers a part of the flow path body 72 and the connection portion 73 of the first pressing cuff 52, a flow path body 82 and three connection portions 83 of a sensing cuff 54, and a flow path body 92 and two connection portions 93 of a second pressing cuff 62 described below. In addition, the cuff cover 33 restricts movement of the covered portion of a part of the first pressing cuff 52, the sensing cuff 54, and the second pressing cuff 62 in a direction away from the bottom portion 31b, thereby fixing the first cuff structure 4 and the second cuff structure 5. Note that the cuff cover 33 may be configured to include a buffer material interposed between the cuff cover and the first cuff structure 4 and between the cuff cover and the second cuff structure 5.

The display unit 12 is disposed directly below the windshield 32. The display unit 12 is electrically connected to the processor 23. The display unit 12 is, for example, a liquid crystal display or an organic electroluminescence display. As a specific example, the display unit 12 is an organic light-emitting diode (OLED). The display unit 12 displays various pieces of information including the date and time, measurement results of blood pressure values such as systolic blood pressure and diastolic blood pressure, heart rate, and the like, and information such as the charge state and amount of charge remaining of the battery 18. The display unit 12 is formed in, for example, the same shape as that of the windshield 32 or a shape slightly smaller than that of the windshield 32 in plan view.

The operation unit 13 is configured to enable input of a command from a user. The operation unit 13 includes, for example, a plurality of buttons 41 provided on the case 11, a sensor configured to detect operation of the buttons 41, and a touch panel 43 provided on the display unit 12 or the windshield 32. When operated by the user, the operation unit 13 converts a command into an electrical signal. The sensor and the touch panel 43 are electrically connected to the processor 23 and output, to the processor 23, electrical signals corresponding to the operations.

The pump 14 is, for example, a piezoelectric pump. The pump 14, for example, compresses air as a fluid and supplies the compressed air through the fluid circuit 24 to an air bag 71 of the first pressing cuff 52 described below and an air bag 81 of the sensing cuff 54 of the first cuff structure 4, and an air bag 91 of the second pressing cuff 62 described below of the second cuff structure 5. The pump 14 is electrically connected to the processor 23.

The acceleration sensor 15 is, for example, a 3-axis acceleration sensor. The acceleration sensor 15, for example, measures acceleration and outputs an analog signal. The acceleration sensor 15 is connected to the processor 23 via, for example, an A/D conversion circuit.

The valve 16 is, for example, an opening and closing valve. The valve 16 opens and closes a fluid circuit connecting the pump 14 to the first cuff structure 4 and/or the second cuff structure 5 and/or a fluid circuit connecting the first cuff structure 4 to the outside (atmosphere) in the fluid circuit 24. The valve 16 is electrically connected to the processor 23. For example, the valve 16 is opened and closed by the control of the processor 23.

As a specific example, the valve 16 is a safety valve configured to release air supplied to the first pressing cuff 52 described below of the first cuff structure 4, the second pressing cuff 62, and the sensing cuff 54 to the atmosphere. For example, when air is to be supplied to the first pressing cuff 52 and the sensing cuff 54 during a blood pressure measurement, the valve 16 is controlled by the processor 23 and thus is switched to the closed state. Further, when the air in the first pressing cuff 52 and the sensing cuff 54 is to be exhausted, the valve 16 is controlled by the processor 23 and thus is switched from the closed state to the open state. Furthermore, the valve 16 may be formed so that the opening degree is adjustable. The valve 16 may be provided on the fluid circuit 24, or may be integrally provided inside a case of the pump 14.

The pressure sensor 17 is provided, for example, in the fluid circuit 24. The pressure sensor 17 detects pressure of the first pressing cuff 52 and/or the sensing cuff 54. For example, the pressure sensor 17 detects pressure of the sensing cuff 54. The pressure sensor 17 is electrically connected to the processor 23 via, for example, an A/D conversion circuit, converts the detected pressure into an electrical signal, and outputs the electrical signal to the processor 23.

The battery 18 is, for example, a secondary battery such as a rechargeable lithium ion battery. The battery 18 is electrically connected to the processor 23. The battery 18 supplies power to the processor 23. The battery 18 supplies driving power to the respective configurations of the processor 23 and via the processor 23 to the display unit 12, the operation unit 13, the pump 14, the acceleration sensor 15, the valve 16, the pressure sensor 17, the communication unit 19, and the biometric sensors 20.

The communication unit 19 is configured to be able to transmit and receive information to and from an external device wirelessly and/or by wire. The communication unit 19 is, for example, a wireless communication module conforming to the wireless communication standards. The communication unit 19, for example, transmits information controlled by the processor 23 and information such as measured blood pressure values and pulses, to an external device, and receives a recording medium for software updates or the like from an external device and transmits the recording medium or the like to a control unit. In the present embodiment, the external device is, for example, an external terminal such as a smart phone, a tablet terminal, a personal computer, or a smart watch.

In the present embodiment, the communication unit 19 and the external terminal may be directly connected, or may be connected over a network. The communication unit 19 and the external terminal may be connected via a mobile communication network such as 4G or 5G, and a wireless communication line such as Wimax or Wi-Fi (trade name). Further, the communication unit 19 and the external device may be connected by wireless communication means such as Bluetooth (trade name) Low Energy (BLE), near field communication (NFC), and infrared communication. Furthermore, the communication unit 19 may include, for example, a general-purpose connector such as a micro-universal serial bus (USB) in addition to a wireless communication module, and a dedicated connector for the blood pressure measurement device 1, and may be connected to the external terminal directly or via a wired communication line such as a local area network (LAN) connection with use of a variety of cables such as a USB cable. Thus, the communication unit 19 may be configured to include a plurality of communication means such as a wireless antenna and a micro-USB connector. Note that a connector for wired communication may be a dedicated connector for the blood pressure measurement device 1.

The biometric sensors 20 are sensors that are in contact with or face the wrist 300 and thus are formed to be able to detect biological information. The biometric sensors 20 each convert the detected biological information into an electrical signal and output the electrical signal to the processor 23. The biometric sensor 20 may be, for example, a sensor configured to measure a physical quantity such as a heart rate or a body temperature, or may be a sensor configured to measure a chemical value such as a blood glucose level or a blood oxygen level. In the present embodiment, the biometric sensors 20 include, for example, a photoplethysmography (PPG) sensor 20a, a percutaneous oxygen saturation (SpO2) sensor 20b, and an electrocardiogram (ECG) sensor 20c.

For example, the PPG sensor 20a measures heart rate by photoplethysmography. For example, the PPG sensor 20a includes a first LED 20d, a second LED 20e, and a first photodiode (PD) 20f.

For example, the SpO2 sensor 20b measures a saturation (percutaneous oxygen saturation). The SpO2 sensor 20b includes the second LED 20e and a second PD 20g. Here, for example, the PPG sensor 20a and the SpO2 sensor 20b share the second LED 20e.

For example, the ECG sensor 20c measures the flow of electricity in the heart to acquire an electrocardiographic waveform. For example, the ECG sensor 20c includes a pair of electrocardiographic electrodes 20c1.

The charging circuit unit 21 includes, for example, an antenna unit 211, a power-receiving unit 212, a charging unit 213, and the charging terminal 214. The charging circuit unit 21 charges the battery 18 by wire power supply and/or wireless power supply. For example, the charging circuit unit 21 receives transmission power transmitted via the antenna unit 211 from an antenna unit 103 of a power transmission device 100 disposed outside and thereby charges the battery 18. In addition, for example, the charging circuit unit 21 receives transmission power transmitted via the charging terminal 214 from a power transmission terminal 104 of the power transmission device 100 disposed outside and thereby charges the battery 18. In other words, the charging circuit unit 21 charges the battery 18 by selectively using the antenna unit 211 and the charging terminal 214 and receiving power from the power transmission device 100. Note that the charging circuit unit 21 may be formed to be capable of both wire power supply and wireless power supply, or may be formed to be capable of only one of wire power supply and wireless power supply.

The antenna unit 211 receives transmission power from the antenna unit 103 of the power transmission device 100. The antenna unit 211 is, for example, a receiver coil as a power-receiving resonance circuit. The antenna unit 211 supplies the received power to the power-receiving unit 212. A power-receiving surface of the antenna unit 211 is formed in a planar shape. The antenna unit 211 is disposed, for example, in the case 11. As a specific example, the antenna unit 211 is disposed adjacent to the display unit 12 on the side opposite to the windshield 32 of the display unit 12 in the case 11. The antenna unit 211 includes, for example, a resonance capacitor and constitutes the power-receiving resonance circuit.

The power-receiving unit 212 rectifies the power received by the antenna unit 211 or the charging terminal 214 and supplies the rectified power to the charging unit 213. As a specific example, the power-receiving unit 212 rectifies the received power supplied from the antenna unit 211 and converts the rectified power from alternating current (AC) to direct current (DC). For example, the power-receiving unit 212 includes a rectifying circuit and a control circuit, controls the operation of the rectifying circuit by the control circuit, and outputs the rectified DC power to the charging unit 213.

The charging unit 213 supplies the power supplied from the power-receiving unit 212 to the battery 18 as charging power. For example, the charging unit 213 converts the power supplied from the power-receiving unit 212 into a predetermined current value and a predetermined voltage value and supplies the converted power to the battery 18. In addition, for example, the charging unit 213 may include a circuit configured to output the charge state of the battery 18 to the power-receiving unit 212 and/or the processor 23.

The charging terminal 214 includes, for example, a pair of terminals 214a, and receives the transmission power from the power transmission terminal 104 of the power transmission device 100 via the pair of terminals 214a.

The memory 22 includes, for example, a random access memory (RAM), a read only memory (ROM), and the like. The memory 22 stores various pieces of data. For example, the memory 22 stores in advance in a changeable manner, for example, various pieces of recording medium data such as recording mediums and applications for controlling the entire blood pressure measurement device 1 and the pump 14, setting data for setting various functions of the blood pressure measurement device 1, calculation data for calculating blood pressure values from the pressure measured by the pressure sensor 17, calculation data for calculating biological information such as a heart rate, a saturation, and an electrocardiographic waveform from the information measured by the biometric sensors 20, and the like so that the data can be altered.

The processor 23 controls the operation of the entire blood pressure measurement device 1 and the operations of the pump 14 and the valve 16 based on the recording mediums stored in the memory 22 and executes a predetermined operation (function). Further, the processor 23 executes the predetermined calculation, analysis, processing, or the like according to the read recording medium. The processor 23 is an arithmetic device such as a central processing unit (CPU). The processor 23 may include, for example, a sub-CPU in addition to a main CPU. Furthermore, the processor 23 displays a state or a result of the executed various operations, calculations, analyses, processing, and the like on the display unit 12 by a recording medium or an application.

The fluid circuit 24 fluidly connects at least two or more of the pump 14, the valve 16, the pressure sensor 17, the first cuff structure 4, and the second cuff structure 5 that are provided in the case 11. The fluid circuit 24 may include, for example, components such as the flow path plate 24a that forms a flow path for the fluid supplied from the pump 14 to the first cuff structure 4, one or a plurality of orifices 24b serving as flow resistance that controls the supply amount and pressure of the fluid supplied to the first cuff structure 4 and/or the second cuff structure 5, and a check valve that controls a flow direction of the fluid. The flow path plate 24a is formed by joining a plurality of plates formed of a resin material or a metal material and sheets or double-sided tape disposed between the plates adjacent to each other and in each of which a slit that forms a flow path is formed. The plurality of orifices 24b may be provided in the flow path plate 24a, or may be provided in a part of the first cuff structure 4 or the second cuff structure 5.

The substrate 25 includes, for example, a control board 25a, a sensor main board 25b, and a sensor sub-board 25c.

On the control board 25a, for example, the acceleration sensor 15, the valve 16, the pressure sensor 17, the communication unit 19, the circuit configuration of the charging circuit unit 21, the memory 22, and the processor 23 are mounted.

On the sensor main board 25b, the first LED 20d, the second LED 20e, the first PD 20f, and the second PD 20g that are used for the PPG sensor 20a and the SpO2 sensor 20b are mounted.

On the sensor sub-board 25c, various circuits and electronic components for configuring the PPG sensor 20a, the SpO2 sensor 20b, and the ECG sensor 20c are mounted. For example, the control board 25a, the sensor main board 25b, and the sensor sub-board 25c are electrically connected to one another.

Hereinafter, a specific example of the first cuff structure 4 will be described with reference to FIG. 1 to FIG. 4 and FIG. 6 to FIG. 8. As illustrated in FIG. 1 to FIG. 4 and FIG. 6 to FIG. 8, the first cuff structure 4 includes a first curler 51, the first pressing cuff 52, a back plate 53, and the sensing cuff 54. The first cuff structure 4 includes, for example, the first curler 51, the first pressing cuff 52, the back plate 53, and the sensing cuff 54. The first cuff structure 4 is configured by sequentially layering the first curler 51, the first pressing cuff 52, the back plate 53, and the sensing cuff 54 toward the wrist 300. Note that the first cuff structure 4 may be configured without including the first curler 51 and/or the back plate 53.

The first curler 51 is fixed, for example, at one end side to the bottom portion 31b, provided on the wrist 300 side, of the case 11. The first curler 51 is formed in a band-like shape that curves in a shape following a circumferential direction of the wrist 300. The first curler 51 consists of a resin material. The first curler 51 is formed of a low hardness material having flexibility and shape retainability. Here, “flexibility” refers to deformation of the shape of the first curler 51 in a radial direction when an external force is applied to the first curler 51 from the band 6. “Shape retainability” refers to the ability of the first curler 51 to maintain a pre-formed shape when no external force is applied to the first curler 51. In other words, the first curler 51 is formed of a resin material with hardness such that no compression deformation is applied or compression deformation is applied but such that elastic deformation such as bending deformation in which the shape, in particular, the curvature of a curved portion changes can be applied. Accordingly, the first curler 51 is formed elastically deformable so as to be bent and deformed by application of an external force and to increase or decrease the internal space in which the wrist 300 is disposed, in accordance with the shape of the wrist to which the device is attached. For example, the first curler 51 is formed of a thermoplastic polyurethane resin (hereinafter, referred to as TPU) or a polypropylene resin.

Further, the first curler 51 is fixed at one end side to the case 11. Furthermore, the first curler 51 is formed to have a length so as to face at least one of two arteries 311, 312 when the blood pressure measurement device 1 is attached to the wrist 300 having the longest circumference among the users who are assumed to wear the device. Preferably, the first curler 51 is formed to have a length so as to face the two arteries 311, 312 when the blood pressure measurement device 1 is attached to the wrist 300 having the longest circumference among the users who are assumed to wear the device. In addition, the first curler 51 is set to a length such that the other end of the first curler 51 does not come into contact with the device body 3 when the blood pressure measurement device 1 is attached to the wrist 300 having the shortest circumference among the users who are assumed to wear the device. The first curler 51 is designed such that, for example, a portion extending from the case 11 of the device body 3 is curved with a predetermined radius of curvature so as to follow the shape of a side of one wrist 300 of the left and right wrists 300 and the shape of a palm side of the wrist 300.

The first pressing cuff 52 is fixed to an inner peripheral surface of the first curler 51 with double-sided tape or an adhesive and by thermal welding or the like. The first pressing cuff 52 is fluidly connected through the fluid circuit 24 to the pump 14. The first pressing cuff 52 is fixed at one main surface to the inner surface of the first curler 51. When being inflated, the first pressing cuff 52 presses the back side of the hand on the wrist 300 and presses the back plate 53 and the sensing cuff 54 toward the wrist 300.

The first pressing cuff 52 includes, for example, one or a plurality of the air bags 71, the flow path body 72 provided at an end portion of the air bag 71, and the connection portion 73 such as a nipple provided in the flow path body 72 and connected to the fluid circuit 24. Here, the air bag 71 is a bag-like structure, and in the present embodiment, the blood pressure measurement device 1 is configured to use air by the pump 14, and thus the present embodiment will be described using the air bag. However, in a case where a fluid other than air is used, the bag-like structure only needs to be any fluid bag that is inflated by the fluid. The air bag 71 is formed in a rectangular bag shape that is long in one direction.

The air bag 71 is formed into a bag shape by performing thermal welding or the like of a plurality of sheet members. For example, when the first pressing cuff 52 is configured to include the plurality of air bags 71, the plurality of air bags 71 are layered, integrally formed by welding or the like, and become fluidly continuous. The sheet members forming the air bag 71 are made of, for example, a thermoplastic elastomer. Here, the thermoplastic elastomer is, for example, TPU.

The flow path body 72 is, for example, integrally provided at a part of one edge portion of the air bag 71 in a longitudinal direction. The flow path body 72 is formed of a portion of two sheet members of the plurality of sheet members forming the air bag 71. In addition, the flow path body 72 is formed in a shape long in one direction and having a width smaller than a width of the air bag 71 in a lateral direction. The flow path body 72 is integrally provided at an end with the connection portion 73. The flow path body 72 is connected via the connection portion 73 to the fluid circuit 24 and constitutes a flow path between the fluid circuit 24 and the air bag 71. A thickness of the flow path body 72 at the time of inflation is smaller than a thickness of the air bag 71 at the time of inflation.

For example, one connection portion 73 is provided. The one connection portion 73 is connected to the fluid circuit 24 via one hole portion 31b3 formed in the bottom portion 31b.

Note that, for example, the flow path body 72 is adjacent to the projection portion 31b1 and extends from one end side to the other end side of the bottom portion 31b in the facing direction of the pair of loop portions 31c so as to avoid the projection portion 31b1 formed at the bottom portion 31b, and the connection portion 73 is connected to one hole portion 31b3 of the three hole portions 31b3 formed at an end portion of the bottom portion 31b on the side where the second cuff structure 5 projects.

The back plate 53 is fixed to the surface of the first pressing cuff 52 on the wrist 300 side with double-sided tape, an adhesive, or the like. The back plate 53 is formed of a resin material. The back plate 53 is formed in, for example, a rectangular plate shape that is long in one direction. Note that the back plate 53 may be configured to be divided, for example, that is, may be formed by aligning a plurality of small rectangular pieces in one direction. The back plate 53 has shape followability.

Here, “shape followability” refers to a function by which the back plate 53 can be deformed so as to follow the shape of a contacted portion of the wrist 300 to be disposed. The contacted portion of the wrist 300 is a region of the wrist 300 that comes into contact with the back plate 53. Here, contact includes both direct contact with the back plate 53 and indirect contact with the back plate 53 via the sensing cuff 54.

The sensing cuff 54 is fixed to a main surface of the back plate 53 on the wrist side. The sensing cuff 54 comes into contact with a region of the wrist 300 where the arteries 311, 312 are present, directly or indirectly via a cover or the like. The sensing cuff 54 is formed into a rectangular shape that is long in one direction. Note that the sensing cuff 54 may be configured to come into contact with a region of the wrist 300 where one of the arteries 311, 312 is present. The sensing cuff 54 is the same size as the first pressing cuff 52 or smaller than the first pressing cuff 52 in a longitudinal direction. In addition, the sensing cuff 54 is the same size as the first pressing cuff 52 or smaller than the first pressing cuff 52 in a lateral direction. The sensing cuff 54 has the same shape as that of the back plate 53 or is smaller than the back plate 53 or is larger than the back plate 53 in a longitudinal direction and a width direction of the back plate 53. The sensing cuff 54 is inflated and thereby compresses the region of the wrist on the palm side where the artery is present. The sensing cuff 54 is pressed by the inflated first pressing cuff 52 toward the living body with the back plate 53 interposed therebetween.

As a specific example, the sensing cuff 54 includes the air bag 81, the flow path body 82 fluidly connected to the air bag 81, and the connection portion 83 such as a nipple provided at the flow path body 82. The air bag 81 and the flow path body 82 are formed into a bag shape made by performing thermal welding or the like of a plurality of sheet members. The sheet members forming the air bag 81 and the flow path body 82 are formed of, for example, a thermoplastic elastomer. Here, the thermoplastic elastomer is, for example, TPU.

Here, the air bag 81 is a bag-like structure and, in the present embodiment, the blood pressure measurement device 1 is configured to use air by the pump 14, and thus the present embodiment will be described using the air bag. However, in a case in which a fluid other than air is used, the bag-like structure may be a fluid bag or the like that is inflated by the fluid. The air bag 81 is formed in a rectangular shape that is long in one direction.

The flow path body 82 is integrally provided, for example, at a part of one edge portion of the air bag 81 in a longitudinal direction. The flow path body 82 is formed by a portion of two sheet members of the plurality of sheet members forming the air bag 81. In addition, the flow path body 82 is formed in a shape long in one direction and having a width smaller than a width of the air bag 81 in a lateral direction. The flow path body 82 is integrally provided at an end with the connection portion 83. The flow path body 82 is connected via the connection portion 83 to the fluid circuit 24 and constitutes a flow path between the fluid circuit 24 and the air bag 81. A thickness of the flow path body 82 at the time of inflation is smaller than a thickness of the air bag 81 at the time of inflation.

For example, three connection portions 83 are provided. The three connection portions 83 are connected to the three hole portions 31b3 formed at an end portion of the bottom portion 31b on the side from which the first cuff structure 4 projects. The three connection portions 83 are connected to the fluid circuit 24.

Hereinafter, a specific example of the second cuff structure 5 will be described with reference to FIG. 1 to FIG. 4 and FIG. 6 to FIG. 8. As illustrated in FIG. 1 to FIG. 4 and FIG. 6 to FIG. 8, the second cuff structure 5 includes a second curler 61 and the second pressing cuff 62. The second cuff structure 5 is configured by sequentially layering the second curler 61 and the second pressing cuff 62 toward the wrist 300.

The second curler 61 is fixed, for example, at one end side to the bottom portion 31b provided on the wrist 300 side of the case 11. The second curler 61 is formed in a band-like shape that curves in a shape following the circumferential direction of the wrist 300. The second curler 61 consists of a resin material. The second curler 61 is formed of a low hardness material having flexibility and shape retainability. Here, “flexibility” refers to deformation of the shape of the second curler 61 in a radial direction when an external force is applied to the second curler 61 from the band 6. “Shape retainability” refers to the ability of the second curler 61 to maintain a pre-imparted shape when no external force is applied to the second curler 61. In other words, the second curler 61 is formed of a resin material with hardness such that no compression deformation is applied or compression deformation is applied but such that elastic deformation such as bending deformation in which the shape, in particular, the curvature of a curved portion changes can be applied. Accordingly, the second curler 61 is formed elastically deformable so as to be bent and deformed by application of an external force and to increase or decrease the internal space in which the wrist 300 is disposed, in accordance with the shape of the wrist to which the device is attached. For example, the second curler 61 is formed of a thermoplastic polyurethane resin (hereinafter referred to as TPU) or a polypropylene resin.

Further, the second curler 61 is fixed at one end side to the case 11. Further, the second curler 61 is formed to have a length so as to face at least one of the two arteries 311, 312 and overlap the first cuff structure 4 at an end portion side when the blood pressure measurement device 1 is attached to the wrist 300 having the longest circumference among the users who are assumed to wear the device. Here, one of the two arteries 311, 312 is the radial artery 311 and the other is the ulnar artery 312. Preferably, the second curler 61 is formed to have a length so as to face the two arteries 311, 312 when the blood pressure measurement device 1 is attached to the wrist 300 having the longest circumference among the users who are assumed to wear the device. Furthermore, the second curler 61 is set to a length such that the other end of the second curler 61 does not come into contact with the device body 3 when the blood pressure measurement device 1 is attached to the wrist 300 having the shortest circumference among the users who are assumed to wear the device. The second curler 61 is designed such that, for example, a portion extending from the case 11 of the device body 3 is curved with a predetermined radius of curvature so as to follow the shape of the side of one wrist 300 of the left and right wrists 300 and the shape of the palm side of the wrist 300.

The second pressing cuff 62 is fixed to an inner peripheral surface of the second curler 61 with double-sided tape or an adhesive and by thermal welding or the like. The second pressing cuff 62 is fluidly connected via the fluid circuit 24 to the pump 14. The second pressing cuff 62 is fixed at one main surface to the inner surface of the second curler 61. When being inflated, the second pressing cuff 62 presses the wrist 300 opposing thereto and presses the overlapped first cuff structure 4 toward the wrist 300. A thickness of the second pressing cuff 62 at the time of inflation is the same as a thickness of the first pressing cuff 52 or larger than the thickness of the first pressing cuff 52 at the time of inflation.

The second pressing cuff 62 includes, for example, one or a plurality of the air bags 91, the flow path body 92 provided at an end portion of the air bag 91, and the connection portion 93 such as a nipple provided in the flow path body 92 and connected to the fluid circuit 24. Here, the air bag 91 is a bag-like structure, and in the present embodiment, the blood pressure measurement device 1 is configured to use air by the pump 14, and thus the present embodiment will be described using the air bag. However, in a case where a fluid other than air is used, the bag-like structure only needs to be any fluid bag that is inflated by the fluid. The air bag 91 is formed in a rectangular bag shape that is long in one direction.

The air bag 91 is formed into a bag shape made by performing thermal welding or the like of a plurality of sheet members. For example, when the second pressing cuff 62 configured to include the plurality of air bags 91, the plurality of air bags 91 are layered, integrally formed by welding or the like, and become fluidly continuous. The sheet members forming the air bag 91 are formed of, for example, a thermoplastic elastomer. Here, the thermoplastic elastomer is, for example, TPU.

The flow path body 92 is, for example, integrally provided at a part of one edge portion of the air bag 91 in a longitudinal direction. The flow path body 92 is formed by a portion of two sheet members of the plurality of sheet members forming the air bag 91. In addition, the flow path body 92 is formed into a shape long in one direction and having a width smaller than a width of the air bag 91 in a lateral direction. The flow path body 92 is integrally provided at one end with the connection portion 93. The flow path body 92 is connected via the connection portion 93 to the fluid circuit 24 and constitutes a flow path between the fluid circuit 24 and the air bag 91. A thickness of the flow path body 92 at the time of inflation is smaller than a thickness of the air bag 91 at the time of inflation.

For example, two connection portions 93 are provided. The two connection portions 93 are connected to the fluid circuit 24 via the two hole portions 31b3 formed in the bottom portion 31b.

Note that, for example, the connection portions 93 are connected to two hole portions 31b3 of the three hole portions 31b3 formed at the end portion of the bottom portion 31b on the side from which the second cuff structure 5 projects.

The first pressing cuff 52 of the first cuff structure 4 and the second pressing cuff 62 of the second cuff structure 5 configured as just described are formed to have lengths such that when the blood pressure measurement device 1 is attached to the wrist 300 having the maximum circumferential length, an inflated portion of the air bag 71 of the first pressing cuff 52 can cover at least one of the two arteries 311, 312 and the air bag 91 of the second pressing cuff 62 can overlap the air bag 71. In addition, the air bag 91 of the second pressing cuff 62 is formed to have a length such that when the blood pressure measurement device 1 is attached to the wrist 300 having the maximum circumferential length and the air bag 91 overlaps the air bag 71 of the first pressing cuff 52, an end edge of the air bag 91 on the side opposite to the flow path body 92 does not overlap the air bag 71 of the first pressing cuff 52 on the artery covered by the air bag 71 of the first pressing cuff 52. In other words, the air bag 91 of the second pressing cuff 62 is set to a length such that when the blood pressure measurement device 1 is attached to the wrist 300 having the maximum circumferential length and the air bag 91 overlaps the air bag 71 of the first pressing cuff 52, the inflated portion of the air bag 91 of the second pressing cuff 62 overlaps the air bag 71 of the first pressing cuff 52 on the artery covered by the air bag 71 of the first pressing cuff 52.

The band 6 is formed into a belt-like shape on an outer peripheral side of the wrist 300 on the side opposite to the first cuff structure 4 and the second cuff structure 5. The band 6 is not fixed to the first cuff structure 4 and the second cuff structure 5. The band 6 is fixed at one end portion to one loop portion 31c. Further, the band 6 includes a pair of hook-and-loop fasteners 6a having a hook formed at one side and a loop formed at the other side. The hook and the loop are engaged with each other, and thus the band 6 with an end portion side inserted into the other loop portion 31c is fixed. Furthermore, the band 6 includes, at the other end portion, a grip 6b that allows a user to easily grip the band.

For example, the band 6 is fixed to the loop portion 31c formed on the side from which the second cuff structure 5 extends, is inserted into the loop portion 31c formed on the side from which the first cuff structure 4 extends, and is then folded back. The band 6 has a length such that when the blood pressure measurement device 1 is attached to the wrist 300 having the maximum circumferential length and to which the device is assumed to be attached, the band 6 can be inserted into the loop portion 31c provided at the outer case 31. In addition, the hook-and-loop fastener 6a is provided at the band 6 while having a length and being arranged such that the band 6 can be folded back and fastened at the wrist 300 having the maximum circumferential length and the minimum circumferential length and to which the device is assumed to be attached. The end portion of the band 6 folded back at the loop portion 31c is pulled in a direction away from the loop portion 31c, and thus, as illustrated in FIG. 3, the blood pressure measurement device 1 is attached to the wrist 300 in a state where the first cuff structure 4 and the second cuff structure 5 are wound around the wrist 300. The band 6 is fixed by the hook-and-loop fasteners 6a, and thus when being inflated, the first pressing cuff 52 and the second pressing cuff 62 are restricted from expanding outward toward the side opposite to the wrist. Therefore, the first pressing cuff 52 and the second pressing cuff 62 can compress the wrist. This allows blood pressure measurement by a known oscillometric method.

The restriction member 7 restricts the relative movement between the first cuff structure 4 and/or the second cuff structure 5 and the band 6 in the width direction. Here, the width direction is a direction orthogonal to the direction in which the band 6 is wound around the wrist 300, in other words, a direction orthogonal to the longitudinal direction of the first cuff structure 4, the second cuff structure 5, and the band 6.

In the present embodiment, the blood pressure measurement device 1 includes the first cuff structure 4 and the second cuff structure 5, and the band 6 is not fixed to the first cuff structure 4 and the second cuff structure 5; therefore, an example in which the restriction member 7 restricts the relative movement among the first cuff structure 4, the second cuff structure 5, and the band 6 in the width direction will be described.

As illustrated in FIG. 2, FIG. 12, and FIG. 13, the restriction member 7 includes a sheet 7a, a first hook-and-loop fastener 7b, a second hook-and-loop fastener 7c, a first restriction portion 7d, and a second restriction portion 7e.

The sheet 7a is formed in a plate shape that is long in one direction. The sheet 7a has a width smaller than the widths of the first cuff structure 4, the second cuff structure 5, and the band 6. The sheet 7a is formed of, for example, a resin material. One end of the sheet 7a is fixed to the outer surface of an end portion of the first cuff structure 4 on the device body 3 side. For example, the sheet 7a is fixed to the outer surface of a portion of the first curler 51 of the first cuff structure 4, which is adjacent to the device body 3. The sheet 7a and the first cuff structure 4 are fixed by, for example, welding, bonding with double-sided tape, an adhesive, or the like, or joining with a hook-and-loop fastener or the like. The sheet 7a is formed to be separable from the first cuff structure 4 with one end as a base point fixed to the first cuff structure 4.

The first hook-and-loop fastener 7b is provided on an outer surface of an end portion (a distal end) of the first cuff structure 4 on the side opposite to the device body 3 side.

The second hook-and-loop fastener 7c is provided, for example, on an inner surface, which is on the wrist 300 side, of an end portion (a distal end) of the sheet 7a on the side opposite to the device body 3 side. The first hook-and-loop fastener 7b and the second hook-and-loop fastener 7c are formed to be connectable. The first hook-and-loop fastener 7b and the second hook-and-loop fastener 7c function as fixing members adapted to fix the sheet 7a to the first cuff structure 4. The fixing member is not limited to the hook-and-loop fastener, and other examples thereof include a magnetic sheet, a snap fit, and the like.

The first restriction portion 7d restricts the relative movement between the first cuff structure 4 and the band 6 in the width direction, and enables a relative movement between the first cuff structure 4 and the band 6 in the longitudinal direction without restricting. In addition, for example, the first restriction portion 7d restricts a relative movement in a direction in which the first cuff structure 4 and the band 6 separate from each other in a direction orthogonal to the width direction and the longitudinal direction of the first cuff structure 4 and the band 6, that is, in a direction along a facing direction of the first cuff structure 4 and the band 6. The first restriction portion 7d engages with the sheet 7a fixed to the first cuff structure 4, thereby restricting the relative movement between the first cuff structure 4 and the band 6 in the width direction and in the direction separating from each other.

For example, the first restriction portion 7d is formed in an annular shape and is formed so that the sheet 7a can be inserted therein. The first restriction portion 7d is provided on an inner surface of the band 6. For example, a sheet-like member is formed into an annular shape and the member is fixed to the inner surface of the band 6, and thus the first restriction portion 7d is formed.

The second restriction portion 7e restricts the relative movement between the first cuff structure 4 and the second cuff structure 5 in the width direction, and enables the relative movement between the first cuff structure 4 and the second cuff structure 5 in the longitudinal direction without restricting. In addition, for example, the second restriction portion 7e restricts a relative movement in a direction in which the first cuff structure 4 and the second cuff structure 5 separate from each other in a direction orthogonal to the width direction and the longitudinal direction of the first cuff structure 4 and the second cuff structure 5, that is, in a direction along a facing direction of the first cuff structure 4 and the second cuff structure 5. The second restriction portion 7e engages with the sheet 7a fixed to the first cuff structure 4, thereby restricting the relative movement between the first cuff structure 4 and the second cuff structure 5 in the width direction and in the direction separating from each other. Thus, the second restriction portion 7e restricts, together with the sheet 7a and the first restriction portion 7d, the movements of the first cuff structure 4, the second cuff structure 5, and the band 6 in the width direction and in the direction in which the first cuff structure 4 and the second cuff structure 5 separate from the band 6 in the facing direction.

For example, the second restriction portion 7e is formed in an annular shape and is formed so that the sheet 7a can be inserted therein. The second restriction portion 7e is provided on the inner surface of the band 6. For example, a sheet-like member is formed into an annular shape and the member is fixed to the inner surface of the band 6, and thus the second restriction portion 7e is formed.

The lengths of the first restriction portion 7d and the second restriction portion 7e in the longitudinal direction of the band 6 and the second cuff structure 5 can be set as appropriate based on the ease of relative movement among the first cuff structure 4, the second cuff structure 5, and the band 6 in the width direction, the posture stabilities of the first cuff structure 4, the second cuff structure 5, and the band 6, and the like. In other words, when the lengths of the first restriction portion 7d and the second restriction portion 7e in the longitudinal direction of the band 6 and the second cuff structure 5 are increased, the contact length of the band 6 and the second cuff structure 5 with the sheet 7a in the width direction can be ensured. Therefore, the sheet 7a can be stably held in the width direction. Also, when the lengths of the first restriction portion 7d and the second restriction portion 7e in the longitudinal direction of the band 6 and the second cuff structure 5 are shortened, the sheet 7a is easily inserted, and friction force in a sliding movement with the sheet 7a can be reduced. Therefore, the first cuff structure 4, the second cuff structure 5, and the band 6 are easily and stably moved relative to each other in the longitudinal direction.

Further, the widths of openings of the first restriction portion 7d and the second restriction portion 7e into which the sheet 7a is inserted are larger than the width of the sheet 7a. The widths of the openings of the first restriction portion 7d and the second restriction portion 7e into which the sheet 7a is inserted can be set as appropriate based on the ease of insertion of the sheet 7a, the ease of relative movement among the first cuff structure 4, the second cuff structure 5, and the band 6 in the width direction, the posture stabilities of the first cuff structure 4, the second cuff structure 5, and the band 6, and the like.

In other words, as a gap between the sheet 7a and the restriction portion 7d and a gap between the sheet 7a and the restriction portion 7e in the width direction are larger, the insertion and movement of the sheet 7a are facilitated, and the relative movement among the first cuff structure 4, the second cuff structure 5, and the band 6 in the width direction is facilitated; however, the relative movable range of the first cuff structure 4, the second cuff structure 5, and the band 6 in the width direction increases. As the gap between the sheet 7a and the restriction portion 7d and the gap between the sheet 7a and the restriction portion 7e in the width direction are smaller, the insertability and movability of the sheet 7a are reduced, and it is difficult for the first cuff structure 4, the second cuff structure 5, and the band 6 to move relative to each other in the width direction. However, the relative movable range of the first cuff structure 4, the second cuff structure 5, and the band 6 in the width direction decreases, and thus the relative movement in the width direction can be suppressed.

As described above, the lengths and widths of the first restriction portion 7d and the second restriction portion 7e can be set according to desired functions or the like. For example, the first restriction portion 7d and the second restriction portion 7e may have the same shape, or the first restriction portion 7d and the second restriction portion 7e may have different shapes, for example, one restriction portion of the first restriction portion 7d and the second restriction portion 7e may be longer and/or wider than the other restriction portion.

The length of the sheet 7a, the position of the pair of hook-and-loop fasteners 7b and 7c, the position of the first restriction portion 7d, and the position of the second restriction portion 7e are set to lengths and/or positions that allow the sheet 7a to be inserted into the first restriction portion 7d and the second restriction portion 7e and allow the sheet 7a and the first cuff structure 4 to be fixed by the hook-and-loop fasteners 7b, 7c when the band 6 is wound around the wrist 300 having the minimum circumferential length and the maximum circumferential length and to which the device is assumed to be attached at the time of attaching the blood pressure measurement device 1 to the wrist 300 having the minimum circumferential length and the maximum circumferential length and to which the device is assumed to be attached. Further, the length of the sheet 7a, the position of the pair of hook-and-loop fasteners 7b and 7c, the position of the first restriction portion 7d, and the position of the second restriction portion 7e are set to lengths and/or positions at which the first cuff structure 4 and the sheet 7a connected to each other by the pair of hook-and-loop fasteners 7b and 7c do not interfere with the second restriction portion 7e in the longitudinal direction of the first cuff structure 4 when the blood pressure measurement device 1 is attached to the wrist 300 having the minimum circumferential length and the maximum circumferential length and to which the device is assumed to be attached. Furthermore, the first restriction portion 7d is disposed at a position in which the first restriction portion 7d does not contact the end portion of the second cuff structure 5 when the blood pressure measurement device 1 is attached to the wrist 300 having the minimum circumferential length and the maximum circumferential length and to which the device is assumed to be attached.

In other words, at the time of attaching the blood pressure measurement device 1 to the wrist 300 having the minimum circumferential length and the maximum circumferential length and to which the device is assumed to be attached, the restriction member 7 enables the first cuff structure 4 and the second cuff structure 5 to be moved to the attachable positions on the wrist 300. In addition, the second restriction portion 7e provided on the band 6 and the second cuff structure 5 do not interfere with each other, and the restriction member 7 does not restrict the relative movement between the second cuff structure 5 and the band 6 in the winding direction on the wrist 300.

As illustrated in FIG. 12 and FIG. 13, such a restriction member 7 releases the connection of the hook-and-loop fastener 7b provided on the first cuff structure 4 and the hook-and-loop fastener 7c provided on the sheet 7a, and allows the sheet 7a to be inserted into the first restriction portion 7d and the second restriction portion 7e in a state where the first cuff structure 4 is separated from the sheet 7a. Then, the hook-and-loop fastener 7c of the sheet 7a inserted into the first restriction portion 7d and the second restriction portion 7e is connected to the hook-and-loop fastener 7b of the first cuff structure 4. Thus, the restriction member 7 restricts the relative movement among the first cuff structure 4, the second cuff structure 5, and the band 6 in the width direction while allowing the relative movement in the longitudinal direction.

Next, an example of a fluid configuration of the device body 3, the first cuff structure 4, the second cuff structure 5, and the fluid circuit 24 of the blood pressure measurement device 1 configured as described above will be described with reference to FIG. 11.

The first pressing cuff 52 is fluidly connected, for example, via the flow path plate 24a to the pump 14. Further, the first pressing cuff 52 and the pump 14 are fluidly connected, for example, via the flow path plate 24a to the valve 16. Furthermore, the first pressing cuff 52 is connected, for example, via the flow path plate 24a to the second pressing cuff 62. In addition, the first pressing cuff 52 is connected to the sensing cuff 54, for example, via the flow path plate 24a and the orifice 24b provided in the flow path plate 24a. The sensing cuff 54 is connected, for example, via the flow path plate 24a to the pressure sensor 17. In addition, the sensing cuff 54 is connected to the atmosphere, for example, via the flow path plate 24a and the orifice 24b provided in the flow path plate 24a. Here, the orifice 24b between the first pressing cuff 52 and the sensing cuff 54 and the orifice 24b between the sensing cuff 54 and the atmosphere are set to flow resistance at which the first pressing cuff 52 and the sensing cuff 54 have a desired difference in pressure.

Next, an example of the power transmission device 100 configured to transmit power to the charging circuit unit 21 of the device body 3 will be described. As illustrated in FIG. 5, the power transmission device 100 includes a power source 101, a power transmission unit 102, the antenna unit 103, and the power transmission terminal 104. Note that the power transmission device 100 may be configured to include one of the antenna unit 103 and the power transmission terminal 104. The power source 101 is, for example, an AC adapter or the like connected to a commercial power source or the like. The power source 101 converts AC power input from the commercial power source into DC power and supplies the DC power to the power transmission unit 102.

The power transmission unit 102 generates AC power as transmission power from the DC power supplied from the power source 101, and supplies the AC power to the antenna unit 103. The power transmission unit 102 generates, for example, the AC power having a frequency that is the same or substantially the same as a resonance frequency of a power transmission resonance circuit generated by the antenna unit 103.

The antenna unit 103 is, for example, a transmitter coil as the power transmission resonance circuit. A power transmission surface of the antenna unit 103 is formed in a planar shape. The antenna unit 103 transmits power to the antenna unit 211 of the device body 3. The antenna unit 103 includes, for example, a resonance capacitor and constitutes a power transmission resonance circuit.

The power transmission terminal 104 comes into contact with the charging terminal 214 provided in the device body 3 and is formed to be fixable to the charging terminal 214.

In the blood pressure measurement device 1 configured as described above, the restriction member 7 can restrict the movement of the first cuff structure 4 and the band 6 in the width direction and restrict the movement of the first cuff structure 4 and the second cuff structure 5 in the width direction. Therefore, the blood pressure measurement device 1 can prevent the first cuff structure 4, the second cuff structure 5, and the band 6 from being displaced in the width direction when the blood pressure measurement device 1 is attached to the wrist 300 and when the first cuff structure 4 and the second cuff structure 5 are inflated. Accordingly, the band 6 can stably press the first cuff structure 4 and the second cuff structure 5 toward the wrist 300, and thus the first cuff structure 4 and the second cuff structure 5 can suitably compress the wrist 300. As a result, the blood pressure measurement device 1 can achieve good measurement accuracy of blood pressure.

Further, the restriction member 7 does not restrict the movement of the first cuff structure 4, the second cuff structure 5, and the band 6 in the longitudinal direction. Thus, both the first cuff structure 4 and the second cuff structure 5 can be moved in the longitudinal direction by winding the band 6 around the wrist 300, and the first cuff structure 4 and the second cuff structure 5 can be suitably wound around the wrist 300. Furthermore, the restriction member 7 is arranged such that the first restriction portion 7d provided on the band 6 and the second cuff structure 5 do not interfere with each other when the blood pressure measurement device 1 is attached to the wrist 300 having the minimum circumferential length and the maximum circumferential length and to which the device is assumed to be attached. Thus, the restriction member 7 does not inhibit the relative movement among the first cuff structure 4, the second cuff structure 5, and the band 6 in the longitudinal direction. As a result, the blood pressure measurement device 1 enables the relative positions among the first cuff structure 4, the second cuff structure 5, and the band 6.

Additionally, in the blood pressure measurement device 1, the first cuff structure 4 and the second cuff structure 5 that extend from the device body 3 and are fluidly connected to each other are respectively disposed on both sides of the device body 3 in one direction. Moreover, the device body 3 includes, at the bottom portion 31b of the case 11, which is the rear surface side, the projection portion (sensor unit) 31b1 as a space in which at least one of the biometric sensors 20 and the charging terminal 214 is disposed at a position separated from the first cuff structure 4 and the second cuff structure 5, for example, on the center side of the bottom portion 31b. Accordingly, the first cuff structure 4, the second cuff structure 5, and at least one of the biometric sensors 20 and the charging terminal 214 can each be disposed on the rear surface side of the device body 3.

Further, the first cuff structure 4 and the second cuff structure 5 are fluidly connected to each other via the fluid circuit 24 including the flow path plate 24a. Note that the first cuff structure 4 and the second cuff structure 5 may directly and fluidly connect the first pressing cuff 52 and the second pressing cuff 62. With these configurations, even when the first cuff structure 4 and the second cuff structure 5 are provided and fixed to the case 11 separately from each other in one direction, the first cuff structure 4 and the second cuff structure 5 can be fluidly connected.

Further, when the blood pressure measurement device 1 is attached to the wrist 300 having the maximum circumferential length and to which the device is assumed to be attached, the air bag 71 of the first pressing cuff 52 covers the region where at least one artery of the two arteries 311, 312 is present, and the air bag 91 of the second pressing cuff 62 can overlap at least a part of the air bag 71 of the first pressing cuff 52.

Accordingly, the wrist 300 can be pressed by the first pressing cuff 52 and the second pressing cuff 62; therefore, even when the air bag 71 of the first pressing cuff 52 and the air bag 91 of the second pressing cuff 62 are not disposed at the rear surface of the device body 3, sufficient pressing force for compressing the artery can be secured.

Thus, when the blood pressure measurement device 1 is attached to the wrist 300 having the maximum circumferential length and to which the device is assumed to be attached, the air bag 71 of the first pressing cuff 52 and the air bag 91 of the second pressing cuff 62 can overlap with a predetermined length. Furthermore, when the blood pressure measurement device 1 is attached to the wrist 300 having the minimum circumferential length, the first cuff structure 4 and the second cuff structure 5 each have a length so as not to come into contact with the device body 3. In addition, the first cuff structure 4 and the second cuff structure 5 are not directly connected to the band 6.

With these configurations, in the blood pressure measurement device 1, the air bag 71 of the first pressing cuff 52 and the air bag 91 of the second pressing cuff 62 are inflated in an overlapping manner; therefore, the artery can be suitably pressed by the first pressing cuff 52 and the second pressing cuff 62. Thus, the blood pressure measurement device 1 is configured such that the biometric sensors 20 can be arranged on the rear surface side of the device body 3, and the blood pressure measurement device 1 can measure blood pressure with high accuracy even when the air bag 71 of the first pressing cuff 52 and the air bag 91 of the second pressing cuff 62 are not disposed on the rear surface of the device body 3.

Further, the case 11 includes the vent 31b6 covered with the waterproof moisture-permeable sheet 31b7 at the bottom portion 31b, thereby allowing ventilation in the case 11. Furthermore, the vent 31b6 and the first cuff structure 4 as well as the second cuff structure 5 are disposed at positions separated from each other in a portion other than the projection portion 31b1 in the bottom portion 31b; therefore, the vent 31b6 can be prevented from being blocked by the first cuff structure 4 and the second cuff structure 5. In addition, the vent 31b6 is provided at a position covered with the cuff cover 33 disposed with a gap from the bottom portion 31b; therefore, the vent 31b6 is protected by the cuff cover 33. As a result, the blood pressure measurement device 1 can prevent dirt from adhering to the vent 31b6 and the waterproof moisture-permeable sheet 31b7, and ventilation function can be prevented from being impaired.

Further, the first cuff structure 4 and the second cuff structure 5 are each configured such that the end portion disposed at the bottom portion 31b of the case 11 is covered with the cuff cover 33, therefore being restricted by the cuff cover 33 from being moved in a direction away from the bottom portion 31b. This makes it possible to prevent the first cuff structure 4 and the second cuff structure 5 from being easily detached from the device body 3.

Furthermore, the blood pressure measurement device 1 is configured such that the various sensors 20a to 20c as the biometric sensors 20 and the charging terminal 214 are provided at the bottom portion 31b of the device body 3. Thus, the device body 3 enables simplification of wiring or the like without needing to allow the biometric sensors 20 and the charging terminal 214 to be provided at a position far from the device body with respect to the substrate 25 provided inside the case 11, which increases flexibility in arrangement. Further, the biometric sensors 20 are provided in the case 11 having high rigidity, and can stably come into contact with the wrist 300 when the blood pressure measurement device 1 is attached to the wrist 300. Therefore, biological information can be suitably acquired by the biometric sensors 20. In addition, the charging terminal 214 is provided at the bottom portion 31b. Accordingly, when the blood pressure measurement device 1 is removed from the wrist 300, the charging terminal 214 is exposed to the outside, and thus charging using the charging terminal 214 is facilitated.

Moreover, in the blood pressure measurement device 1, the first pressing cuff 52 and the second pressing cuff 62 are fluidly connected via the fluid circuit 24 provided inside the device body 3, and thus the arrangement of the first pressing cuff 52 and the second pressing cuff 62 at the bottom portion 31b can be simplified.

In the blood pressure measurement device 1 configured as described above, the restriction member 7 enables the movement of the first cuff structure 4, the second cuff structure 5, and the band 6 in the longitudinal direction and restricts the movement in the width direction. Thus, the relative positions among the first cuff structure 4, the second cuff structure 5, and the band 6 can be easily adjusted, and the first cuff structure 4, the second cuff structure 5, and the band 6 can be prevented from being displaced in the width direction.

Note that the present invention is not limited to the embodiment described above. For example, in the example described above, the configuration is described in which the first pressing cuff 52 of the first cuff structure 4 and the second pressing cuff 62 of the second cuff structure 5 are connected, for example, to the flow path plate 24a of the fluid circuit 24 via the flow path bodies 72 and 92 and the connection portions 73 and 93 respectively formed, and are connected fluidly in the fluid circuit 24.

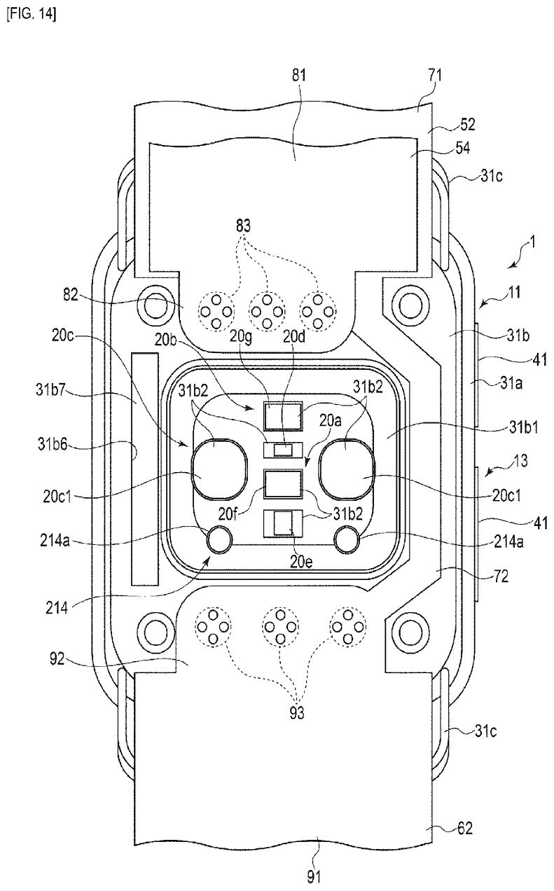

However, the first pressing cuff 52 and the second pressing cuff 62 may be directly and fluidly connected. For example, as illustrated in FIG. 14, the first cuff structure 4 and the second cuff structure 5 may be configured such that the flow path body 72 of the first pressing cuff 52 and the flow path body 92 of the second pressing cuff 62 are fluidly and integrally continuous and are connected to the fluid circuit 24 by the connection portions 93 formed on the flow path body 72 or the flow path body 92. With such a configuration, the first pressing cuff 52 and the second pressing cuff 62 are connected while avoiding the projection portion 31b1 that is the sensor unit, and thus the first pressing cuff 52 and the second pressing cuff 62 can be prevented from interfering with the projection portion 31b1 and inhibiting the functions of the biometric sensors 20 and the charging terminal 214 that are disposed on the projection portion 31b1.

In the example described above, the configuration is described in which the first cuff structure 4 includes the first curler 51, the first pressing cuff 52, the back plate 53, and the sensing cuff 54 and the second cuff structure 5 includes the second curler 61 and the second pressing cuff 62.

However, the first cuff structure 4 may be configured not to include at least one of the first curler 51 or the back plate 53, and the second cuff structure 5 may be configured not to include the second curler 61. In a case where the first cuff structure 4 does not include the first curler 51, the sheet 7a and the first hook-and-loop fastener 7b of the restriction member 7 only need to be provided on the outer surface of the distal end on the side opposite to the flow path body 72 of the air bag 71 of the first pressing cuff 52.

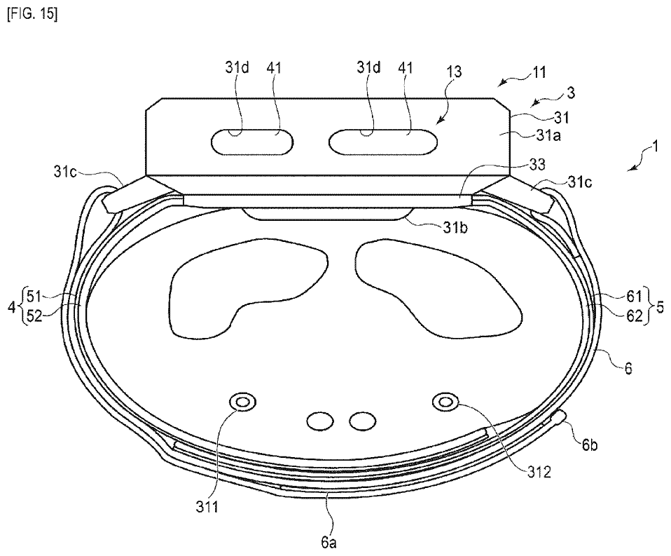

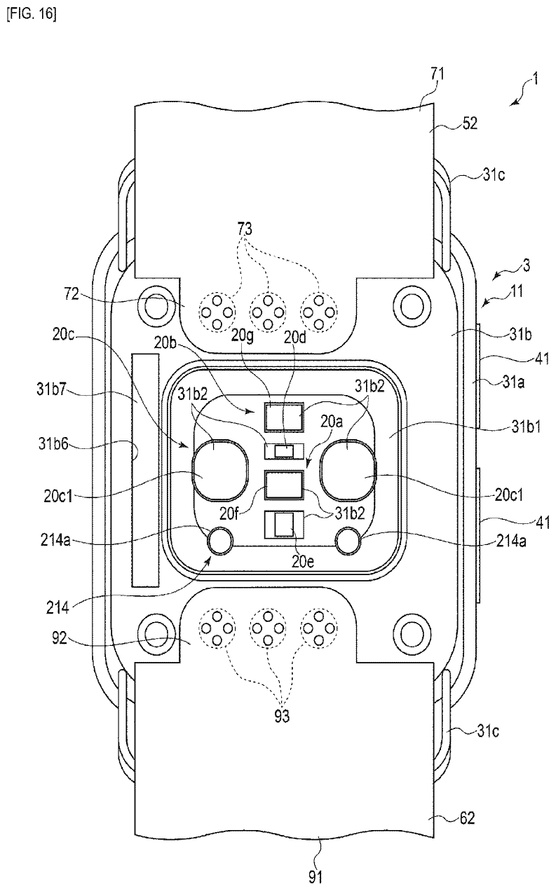

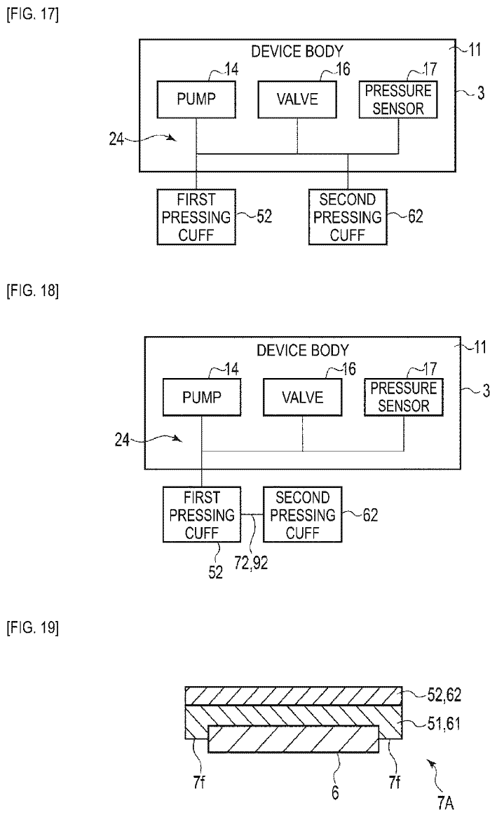

Further, as illustrated in FIG. 14 and FIG. 15, the first cuff structure 4 may be configured not to include the sensing cuff 54. For example, in a case where the first cuff structure 4 does not include the sensing cuff 54, as illustrated in FIG. 16 and FIG. 17, the pressure sensor 17 may be fluidly connected at least to the first pressing cuff 52, and blood pressure may be obtained from the pressure of the first pressing cuff 52. Furthermore, even in a case where the sensing cuff 54 is not provided, the first pressing cuff 52 and the second pressing cuff 62 may be fluidly connected to each other via the fluid circuit 24 in the device body 3 as illustrated in FIG. 17, and, for example, the flow path body 72 and the flow path body 92 may be fluidly connected to each other as illustrated in FIG. 18.

Further, in the example described above, an example in which the band 6 is not fixed to the first cuff structure 4 and the second cuff structure 5 is described. However, the band 6 may be fixed to the second cuff structure 5 located on the outer side when the first cuff structure 4 and the second cuff structure 5 are overlapped. In other words, even when the band 6 is fixed to the second cuff structure 5, the first cuff structure 4 located closer to the wrist 300 than the second cuff structure 5 is located is not fixed to the band 6. In such a case, by tightening the band 6, the first cuff structure 4 and the second cuff structure 5 can be tightened and brought into close contact with the wrist 300.

Furthermore, in the example described above, the device body 3 is provided with the PPG sensor 20a, the SpO2 sensor 20b, and the ECG sensor 20c as the biometric sensors 20 at the bottom portion 31b of the outer case 31, which forms the rear surface side of the case 11. However, the biometric sensors 20 may be any one of the PPG sensor 20a, the SpO2 sensor 20b, and the ECG sensor 20c, or may include a sensor that acquires other biological information, in addition to these sensors 20a to 20c or in place of these sensors 20a to 20c.

Additionally, in the example described above, an example is described in which the blood pressure measurement device 1 includes, as a pair of overlapping cuffs, the first cuff structure 4 and the second cuff structure 5 that can overlap the first cuff structure 4, but no such limitation is intended. For example, the blood pressure measurement device 1 may include the device body 3, the first cuff structure 4, the band 6, and the restriction member 7 without including the second cuff structure 5. In such a blood pressure measurement device 1, the restriction member 7 need not include the second restriction portion 7e.

Moreover, in the example described above, the restriction member 7 includes the sheet 7a, the pair of hook-and-loop fasteners 7b and 7c, the first restriction portion 7d, and the second restriction portion 7e, but no such limitation is intended. In other words, the restriction member 7 only needs to restrict the movement of the first cuff structure 4 and/or the second cuff structure 5 and the band 6 in the width direction and allow the movement in the longitudinal direction. For example, as in a restriction member 7A of another example illustrated in FIG. 19, protrusions 7f contactable with side surfaces of the band 6 in the width direction may be provided at both ends of the first curler 51 and/or the second curler 61 of the first cuff structure 4 and/or the second cuff structure 5 in the width direction, and the protrusions 7f and the side surfaces of the band 6 may be brought into contact with each other. A protrusion provided on one of the first curler 51 and/or the second curler 61 and the band 6 and an opening provided on the other may constitute the restriction member. In other words, the restriction member only needs to include a member or a portion contactable with the first cuff structure 4 and/or the second cuff structure 5 and the band 6 in the width direction.

In other words, the present invention is not limited to the embodiments described above, and various modifications can be made in an implementation stage without departing from the scope of the invention. Further, the embodiments may be carried out as appropriate in combination to the extent possible, and combined effects can be obtained in such a case. Furthermore, the inventions at various stages are included in the embodiments described above, and the various inventions can be extracted in accordance with appropriate combinations in a plurality of disclosed constituent elements. Note that the present invention is not limited to the embodiments described above, and various modifications can be made in an implementation stage without departing from the scope of the invention. Further, the embodiments may be carried out as appropriate in combination, and combined effects can be obtained in such a case. Furthermore, the various inventions are included in the embodiment, and the various inventions may be extracted in accordance with combinations selected from the plurality of disclosed constituent elements. For example, in a case where the problem can be solved and the effects can be obtained even when some of the constituent elements are removed from the entire constituent elements given in the embodiment, the configuration obtained by removing the constituent elements may be extracted as an invention.

### REFERENCE NUMERALS LIST

1 Blood pressure measurement device
3 Device body
4 First cuff structure
5 Second cuff structure
6 Band
6a Hook-and-loop fastener
6b Grip
7 Restriction member
7a Sheet
7b First hook-and-loop fastener (fixing member)
7c Second hook-and-loop fastener (fixing member)
7d First restriction portion
7e Second restriction portion
7f Protrusion
11 Case
12 Display unit
13 Operation unit
14 Pump
15 Acceleration sensor
16 Valve
17 Pressure sensor
18 Battery
19 Communication unit
20 Biometric sensor
20a PPG sensor
20b SpO2 sensor
20c ECG sensor
20c1 Electrocardiographic electrode
20d First LED
20e Second LED
20f First PD
20g Second PD
21 Charging circuit unit
22 Memory
23 Processor
24 Fluid circuit
24a Flow path plate
24b Orifice
25 Substrate
25a Control board
25b Sensor main board
25c Sensor sub-board
31 Outer case
31a Peripheral wall portion
31b Bottom portion (rear surface)
31b1 Projection portion (sensor unit)
31b1 Sensor unit
31b2 Window portion
31b3 Hole portion
31b4 Opening
31b5 Cover
31b6 Vent
31b7 Waterproof moisture-permeable sheet
31c Loop portion
31d Opening
32 Windshield
33 Cuff cover
33a Opening
33b Hole
41 Button
43 Touch panel
51 First curler
52 First pressing cuff
53 Back plate
54 Sensing cuff
61 Second curler
62 Second pressing cuff
71 Air bag
72 Flow path body
73 Connection portion
81 Air bag
82 Flow path body
83 Connection portion
91 Air bag
92 Flow path body
93 Connection portion
100 Power transmission device
101 Power source
102 Power transmission unit
103 Antenna unit
104 Power transmission terminal
211 Antenna unit
212 Power-receiving unit
213 Charging unit
214 Charging terminal
214a Terminal
300 Wrist
311 Radial artery
312 Ulnar artery

What is claimed is:

1. A blood pressure measurement device comprising:
a device body;
a first cuff provided at the device body and configured to press a living body;
a band provided on an outer surface side of the first cuff and wound around the living body; and
a restriction member configured to restrict a relative movement between the first cuff and the band in a width direction and to allow a relative movement between the first cuff and the band in a longitudinal direction, wherein the restriction member includes:
a sheet fixed to the first cuff on a side close to the device body, the sheet being separable from a distal end side of the first cuff;
fixing members provided at a portion in which the sheet and the first cuff face each other, the fixing members being configured to fix the sheet and the first cuff; and
a first restriction portion provided on the band and into which the sheet is insertable, the first restriction portion being configured to restrict movement of the sheet in a width direction.

2. The blood pressure measurement device according to claim 1, wherein the sheet, the fixing members, and the first restriction portion are formed to have lengths and/or positions that allow the sheet to be inserted into the first restriction portion and allow the sheet and the first cuff to be fixed by the fixing members when the band is wrapped around the living body having a minimum circumferential length and a maximum circumferential length and to which the device is assumed to be attached.

3. A blood pressure measurement device comprising:
a device body;
a first cuff provided at the device body and configured to press a living body;
a band provided on an outer surface side of the first cuff and wound around the living body;
a restriction member configured to restrict a relative movement between the first cuff and the band in a width direction and to allow a relative movement between the first cuff and the band in a longitudinal direction; and
a second cuff that can be overlapped with the first cuff on an outer side of the first cuff, wherein the restriction member restricts a relative movement between the second cuff and at least one of the first cuff or the band in the width direction.

4. The blood pressure measurement device according to claim 3, wherein the restriction member includes:
a sheet fixed to the first cuff on a side close to the device body, the sheet being separable from a distal end side of the first cuff;
fixing members provided at a portion in which the sheet and the first cuff face each other;
a first restriction portion provided on an inner surface of the band and into which the sheet is insertable, the first restriction portion being configured to restrict movement of the sheet in the width direction; and
a second restriction member provided on an inner surface of the second cuff and into which the sheet is insertable, the second restriction portion being configured to restrict movement of the sheet in the width direction.

5. The blood pressure measurement device according to claim 4, wherein the sheet, the fixing members, the first restriction portion, and the second restriction portion are formed to have lengths and/or positions that allow the sheet to be inserted into the first restriction portion and the second restriction portion and allow the sheet and the first cuff to be fixed by the fixing members when the band is wrapped around the living body having a minimum circumferential length and a maximum circumferential length and to which the device is assumed to be attached.
